SEQUENCE LISTING (12 sequences)

SEQ ID NO: 1 - protein, 115 aa, synthetic construct
EVQLQQSGPELVKPGASVKISCKTSGYTFTEYTMHWVRQSHGKSLEWIGGIDRYNGDTYYNQKFKDKATLTVDRSSSTAYMELRSLTSEDSAVYFCARNGAMDYWGQGISVTVSS

SEQ ID NO: 2 - protein, 132 aa, synthetic construct
MKLPVRLLVLMFWIPASSSDVVMTQTPLSLPVSLGDQASISCRSSQSLVHSNGNTYLHWFLQKPGQSPKLLIYKVSIRFSGVPDRFSGSGSGTDFTLKISRVEAEDLGVYFCSQSTHVPPLTFGAGTKLELK

SEQ ID NO: 3 - protein, 445 aa, synthetic construct
EVQLQQSGPELVKPGASVKISCKTSGYTFTEYTMHWVRQSHGKSLEWIGGIDRYNGDTYYNQKFKDKATLTVDRSSSTAYMELRSLTSEDSAVYFCARNGAMDYWGQGISVTVSSATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 4 - protein, 220 aa, synthetic construct
DVVMTQTPLSLPVSLGDQASISCRSSQSLVHSNGNTYLHWFLQKPGQSPKLLIYKVSIRFSGVPDRFSGSGSGTDFTLKISRVEAEDLGVYFCSQSTHVPPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 5 aa, synthetic construct
EYTMH

SEQ ID NO: 6 - protein, 17 aa, synthetic construct
GIDRYNGDTYYNQKFKD

SEQ ID NO: 7 - protein, 6 aa, synthetic construct
NGAMDY

SEQ ID NO: 8 - protein, 16 aa, synthetic construct
RSSQSLVHSNGNTYLH

SEQ ID NO: 9 - protein, 7 aa, synthetic construct
KVSIRFS

SEQ ID NO: 10 - protein, 10 aa, synthetic construct
SQSTHVPPLT

SEQ ID NO: 11 - protein, 147 aa, synthetic construct
MKFLVNVALVFMVVYISFIYASMEECWVTEIANGSKDGLDSNPMKDYMILSGPQKAAADYKDDDDKLSPIQHDFPALVMILIILGVMAGIIGTILLISYCISRMTKKSSVDIQSPEGGDNSVPLSSIEQTPNEESSNVSGGHHHHHH

SEQ ID NO: 12 - protein, 147 aa, synthetic construct
MKFLVNVALVFMVVYISFIYASMEGCRETEVTNGSNGGLEFNPMKEYMILSSGQQAAADYKDDDDKLSPIQHDFPALVMILIILGVMAGIIGTILLISYCISRMTKKSSVDIQSPEGGDNSVPLSSIEQTPNEESSNVSGGHHHHHH

(19) United States
(12) Patent Application Publication
(10) Pub. No.: US 2026/0029420 A1
(43) Pub. Date: Jan. 29, 2026

(54) ANTI-CB2 ANTIBODIES AND THEIR USE IN A FLOW CYTOMETRY ASSAY TO MEASURE CELL SURFACE CB2 EXPRESSION

(71) Applicant: Hoffmann-La Roche Inc., Little Falls, NJ (US)

(72) Inventors: Katrijn BOGMAN, Folgensbourg (FR); Barbara ECABERT, Therwil (CH); Andreas GUENTHER, Steinen (DE); Ulrich Friedrich Oskar LUHMANN, Riehen (CH)

(73) Assignee: Hoffmann-La Roche Inc., Little Falls, NJ (US)

(21) Appl. No.: 19/066,127

(22) Filed: Feb. 27, 2025

Related U.S. Application Data
(63) Continuation of application No. PCT/EP2023/073742, filed on Aug. 30, 2023.

(30) Foreign Application Priority Data
Sep. 1, 2022 (EP) 22193362.5

(51) Int. Cl. G01N 33/94; C07K 16/28; G01N 33/50

(52) U.S. Cl. CPC G01N 33/948; C07K 16/28; G01N 33/5091; C07K 2317/24; G01N 2800/52

(57) ABSTRACT

The present invention relates to novel antibodies which bind to human CB2. Further, the invention relates to a method of detecting changes in cell surface CB2 expression by flow cytometry in response to the treatment with a CB2 agonist. The method allows to assess and monitor target engagement of CB2 agonists thus supporting pre-clinical and clinical development of CB2 agonists.

### CROSS-REFERENCE TO RELATED APPLICATIONS

This application is a continuation of International Application No. PCT/EP2023/073742, filed Aug. 30, 2023, which claims benefit of priority to European Application No. 22193362.5, filed Sep. 1, 2022, each of which is incorporated herein by reference in its entirety.

### SEQUENCE LISTING

The instant application contains a sequence listing which has been submitted electronically in.xml format and is hereby incorporated by reference in its entirety. Said.xml copy, created on Feb. 18, 2025, is named P37689-US_Sequence_Listing.xml and is 12,436 bytes in size.

### FIELD OF THE INVENTION

The present invention relates to novel antibodies which specifically bind to human Cannabinoid Receptor 2 (CB2) and their use in in vitro pre-clinical assays as well as in clinical trials to monitor CB2 agonist induced CB2 internalization as a target engagement biomarker.

### BACKGROUND OF THE INVENTION

Frequently, monoclonal antibodies (mAbs) are raised against synthetic peptides derived from the predicted sequence of the target protein. Unfortunately, these antibodies, though strongly reactive with the peptide, frequently fail to recognize the native protein. Another standard procedure to generate mAbs uses purified recombinantly expressed proteins. Procaryotic expression systems are the most widely used expression hosts. But when studying mammalian surface proteins it is often necessary to use mammalian expression systems, as they are more likely to produce functional proteins with the appropriate disulfide-bonds and posttranslational modifications. Although introduction of affinity tags simplifies purification, it often remains difficult to obtain recombinant protein in native conformation and in sufficient yield and purity. This applies most notably to membrane and membrane associated protein, as they are likely to lose their native structure during the purification processes.

When attempting to generate mAbs capable of recognizing the native protein, it is also critical to use the target protein in its native confirmation not only in the immunization step but also for the screening procedure. Many standard hybridoma-screening protocols make use of recombinant proteins immobilized on solid supports, which may significantly alter protein confirmation.

With the objective of generating mABs specifically recognizing membrane-associated proteins in their native confirmation, we applied a methodology that bypasses any need for purified recombinant protein. This strategy utilizes antigens expressed on the surface of stably transfected mammalian cells both for immunization of mice and for immunoassays, such as testing seroconversion, hybridoma selection and mAB characterization (Dreyer A. et al. BMC Biotechnology 2010, 10:87).

The cannabinoid receptors are a class of cell membrane receptors belonging to the G protein-coupled receptor superfamily. There are currently two known subtypes, termed Cannabinoid Receptor 1 (CB1) and Cannabinoid Receptor 2 (CB2). CB1 is mainly expressed in the central nervous system (i.e. amygdala cerebellum, hippocampus) and to a lesser amount in the periphery (liver, skeletal muscle, pancreas, and fat). CB2 is encoded by the CNR2 gene and is mostly expressed peripherally on cells of the immune system such as macrophages and other subtypes of leukocytes (Ashton, J. C. et al. Curr Neuropharmacol 2007, 5(2), 73-80; Miller, A. M. et al. Br J Pharmacol 2008, 153(2), 299-308; Centonze, D., et al. Curr Pharm Des 2008, 14(23), 2370-42). Interestingly, CB2 expression levels on mRNA levels vary between immune cell populations in both resting and activation state indicating potentially an important role for CB2 in immune regulation and function. Moreover, the CB2 receptor is also expressed in the gastrointestinal system (Wright, K. L. et al. Br J Pharmacol 2008, 153(2), 263-70) and is widely distributed in the brain where it is found primarily on microglia and not neurons (Cabral, G. A. et al. Br J Pharmacol 2008, 153(2): 240-51).

The discovery of the endocannabinoid system (ECS) in the central nervous system and in various peripheral organs has prompted nonclinical studies to explore its role in health and disease. The results of these studies have implicated the ECS in a variety of pathophysiological processes (Pacher, P. et al. FEBS J 2013; 280:1918-43).

CB1 is known to be responsible for the psychoactive effects of cannabinoids yet in the peripheral tissues receptor activation displays potent oxidative, inflammatory, and profibrotic activity. In contrast, CB2 activation has been shown to protect against tissue damage (Cabral, G. A. et al. Expert Rev Mol Med 2009; 11: e3). For example, in animal models of chronic degenerative diseases such as neurodegenerative inflammatory disorders, atherosclerosis, and liver fibrosis activation of CB2 has been shown to reduce inflammatory, oxidative, and fibrotic processes. Interestingly, in animal models diabetic and other nephropathies can be treated by CB2 agonism (Behl, T. et al. Diabetes Metab Res Rev 2016; 32:251-9). Furthermore, activation of CB2 has been shown to be anti inflammatory in the eye (Xu, H. et al. J Leukoc Biol 2007;82:532-41; Toguri, J. T. et al. Br J Pharmacol 2014;171:1448-61).

These data indicate that modulation of the endocannabinoid system with an agonistic approach may have a multitude of potential benefits in acute, sub-chronic and chronic conditions due its anti-inflammatory, anti-fibrotic and vascular health promoting effects. For example, CB2 agonists may be beneficial in the treatment of microvascular diabetic complications such as diabetic retinopathy (DR) and diabetic macular edema (DME) (Gruden, G. et al. Br J Pharmacol 2016;173:1116-27).

The CB2 is member of the G-protein coupled receptor family which upon ligand binding can respond by internalization of the receptor into the endosomal compartment of the cell, thereby removing the respective receptor form the cell surface and initiating downstream signaling effects (Calebiro, D. et al. Best Pract Res Clin Endocrinol Metab 2018:32(2): 83-91). Thus, cell surface expression of CB2 may be suitable as biomarker of CB2 agonist target engagement. There is a need for sensitive and specific assays to detect changes in cell surface expression of CB2 to enable drug effect monitoring in vitro, in vivo and in the clinical setting and support linking CB2 target engagement with downstream PD effects and clinical effects in the respective indications.

### SUMMARY OF THE INVENTION

The invention disclosed herein provides an antibody comprising an antigen binding moiety that binds to human cannabinoid receptor subtype 2 (CB2), wherein the antibody comprises a heavy chain variable region (VH) comprising a heavy chain complementary determining region (HCDR) 1 of SEQ ID NO: 5, a HCDR 2 of SEQ ID NO: 6, and a HCDR 3 of SEQ ID NO: 7, and a light chain variable region (VL) comprising a light chain complementarity determining region (LCDR) 1 of SEQ ID NO: 8, a LCDR 2 of SEQ ID NO: 9 and a LCDR 3 of SEQ ID NO: 10. In one aspect, the antibody comprises a heavy chain variable region (VH) comprising an amino acid sequence that is at least about 95%, 96%, 97%, 98%, 99% or 100% identical to the amino acid sequence of SEQ ID NO: 1, and (b) a light chain variable region (VL) comprising an amino acid sequence that is at least about 95%, 96%, 97%, 98%, 99% or 100% identical to the amino acid sequence of SEQ ID NO: 2.

In one aspect, the antibody comprises an Fc domain. In a further aspect, the Fc domain is an IgG class, particularly an IgG₃ subclass, Fc domain. In yet another aspect, the Fc domain is a murine Fc domain.

In another aspect, the antibody comprises a polypeptide sequence that is at least about 95%, 96%, 97%, 98%, 99% or 100% identical to the amino acid sequence of SEQ ID NO: 3 and a polypeptide sequence that is at least about 95%, 96%, 97%, 98%, 99% or 100% identical to the amino acid sequence of SEQ ID NO: 4.

In one embodiment, the antibody cross-reacts with cynomolgus CB2.

Further, the invention provides a method for detecting changes in cell surface CB2 expression in response to treatment with a CB2 agonist by flow cytometry in biological samples obtained from a subject, the method comprising: a) detecting cell surface CB2 expression levels in a reference sample that has not been treated with a CB2 agonist, b) detecting cell surface CB2 expression in a sample after treatment with a CB2 agonist, and c) comparing the cell surface CB2 expression levels of step b) to those of step a), wherein a decrease in cell surface CB2 expression in response to CB2 agonist treatment is indicative of target engagement, wherein cell surface CB2 expression is detected using an antibody according to the invention disclosed herein.

Moreover, a method of assessing treatment response to a CB2 agonist in a subject by flow cytometry, is provided, the method comprising: a) detecting cell surface CB2 expression levels in a biological sample obtained from said subject before treatment with the CB2 agonist, b) detecting cell surface CB2 expression levels in a biological sample obtained from said subject after treatment with the CB2 agonist, and c) comparing the cell surface CB2 expression levels of step b) to those of step a), wherein cell surface CB2 expression is detected using an anti-human CB2 antibody according to the invention disclosed herein.

In one aspect, the cell surface expression of CB2 in the afore-mentioned methods is detected by a) a detection moiety which is conjugated to the anti-human CB2 antibody, or b) a second antibody comprising a detection moiety which binds to the anti-human CB2 antibody.

In one aspect, the CB2 agonist used in the methods is (S)-1-[5-tert-Butyl-3-(1-methyl-1H-tetrazol-5-ylmethyl)-3H-[1,2,3]triazolo [4,5-d]pyrimidin-7-yl]-pyrrolidin-3-ol. In another aspect, the subject is human or cynomolgus monkey.

In one aspect, the biological samples are treated in vitro with the CB2 agonist. While in another aspect, the subject was treated with the CB2 agonist. In one aspect, the biological samples are whole blood.

In a further aspect, a dose-response relationship of a CB2 agonist is determined. In a further aspect, the dose-response relationship of a study population supports the clinical dose selection for a target population.

Moreover, an antibody according to the invention for use in an afore-mentioned method is provided.

Also encompassed by the invention is the use of an antibody according to the invention in an afore-mentioned method.

### BRIEF DESCRIPTION OF THE FIGURES

FIGS. 1A-1C: Specificity of CB2 antibodies analyzed by Western blot. FIG. 1A: Western blot images for antibodies hCB2-1/622, hCB2-2/9 and mCB2-1/45, PBS and comparison with total protein colloidal blue staining. Bottom row shows blots with longer exposure time compared to top row. FIG. 1B: Molecular weight marker (MWM) used in Western Blot experiment. FIG. 1C: Sample loading scheme of Western Blots and sample description.

FIGS. 2A-2D: Specificity of CB2 antibodies analyzed by Immunoprecipitation. FIG. 2A: Western Blot analysis of Samples after immune precipitation with respective anti-human CB2 antibodies or PBS and Western blot of the total cell lysate (WB) (Detection: hCB2-2/9-HRPO). Arrows indicate human CB2 band. FIG. 2B: Immune precipitation analysis with a mouse specific CB2 antibody relative to the input sample confirms specificity of the mouse antibody (Detection: mCB2-1/45-HRPO). Arrow indicates mouse CB2 band. FIG. 2C: Molecular weight marker (MWM) used in Immunoprecipitation experiment. FIG. 2D: Sample loading scheme and sample description.

FIG. 3: Analysis of the specificity of the anti hCB2 antibody 1/622 for hCB2. Flow cytometric analysis with increasing concentrations of hCB2 antibody 1/622 in CHO cells transfected with hCB2 (light brick) and hCB1 (dark brick). Untransfected CHO cells (black) serve as a negative control. For detection an F(ab)₂ goat-anti Mouse IgG₃ PE antibody was used.

FIG. 4: Flow cytometric analysis of hCB2 antibody 1/622 in CHO-DUKX parental and CHO-DUKX_HOMSA_CNR2_Clone_90_CRE-Luc cells transfected with hCB2. For detection an F(ab)₂ goat-anti Mouse IgG₃ PE antibody was used. The anti-human CB2 antibody 1/622 detects the CB2 in the CHO cells transfected with hCB2 with a signal to background ratio of 10, the CHO-DUKX parental cells are negative.

FIG. 5A and FIG. 5B: Comparison of different anti-hCB2 antibodies in detecting cell surface CB2 expression on B-cells. FIG. 5A: Flow cytometric analysis with increasing concentrations of hCB2 antibody 1/622 (black dots) and hCB2 clone 352110 (R&D Systems; dark rectangle). A fluorescence minus one (FMO) serves as control as well as the secondary PE labeled antibody alone (0 μg antibody/sample). FIG. 5B: Flow cytometric analysis with increasing concentrations of mouse IgG₃ isotype control clone B10 (SBA; open dots), hCB2 antibody 1/622 (black dots), mouse IgG_{2b} isotype control clone 26114 (R&D Systems; open rectangles) and hCB2 clone 352110 (R&D Systems; dark rectangles). The secondary PE labeled antibody alone serves as 0 μg antibody/sample control.

FIGS. 6A-6F: Example of a gating strategy of the flow cytometry assay for CB2 detection on T-and B-cells. FIG. 6A: Single cells are identified to exclude forward scatter doublets. FIG. 6B: From the single cell population the CD45⁺ lymphocytes are identified. FIG. 6C: From the CD45⁺ lymphocyte population the PBMCs are identified. FIG. 6D: From the PBMC population CD3⁺ CD14⁻ T-cells and CD3⁻ CD14⁻ cells are identified. FIG. 6E: From the CD3⁻ CD14⁻ gate the CD3⁻ CD19⁺ B-cells are identified. FIG. 6F: CB2 PE is plotted in a histogram on CD3⁻ CD19⁺ B-cells.

FIG. 7: IC50 determination for CB2 internalization on B-cells in response to RG7774 in whole blood of healthy participants. Human whole blood was incubated with RG7774 for 1 hour at 37° C. followed by incubation with the anti-human CB2 antibody 1/622. The expression of CB2 on the surface of B-cells was analyzed using a lineage marker mix specific for B-cells (CD45⁺ CD3⁻ CD14⁻ CD19⁺) and T-cells (CD45⁺ CD3⁺ CD14⁻). The B/T-cell CB2 MFI ratio was calculated and normalized to baseline. 0 μM was set as 100% baseline receptor expression.

FIGS. 8A-8E: Baseline median fluorescence intensity (MFI)±SD of CB2 surface expression on different cell sub-populations in whole blood from diabetic patients (DP; triangles, type 1 or type 2) and matched healthy participants (HP; dot) at baseline. FIG. 8A: Average baseline MFI on B-cells (CD45⁺ CD3⁻ CD14⁻ CD19⁺). FIG. 8B: Average baseline MFI on Basophils (CD45⁺ CD3⁻ CD14⁻ CD19⁻ CD193⁺ IgE⁺). FIG. 8C: Average baseline MFI on Monocytes (CD45⁺ CD3⁻ CD14⁺). FIG. 8D: Average baseline MFI on T-cells (CD45⁺ CD3⁺ CD14⁻). FIG. 8E: Average baseline MFI on NK cells (CD45⁺ CD3⁻ CD14⁻ CD19⁻ CD193⁻ IgE⁻ CD16⁺ CD56⁺). Mean MFI for individual data points is based on duplicate measurements. * indicates significant change using Oneway ANOVA analysis (p=0.0245 with Tukey multiple testing correction (p<0.05). n (HPs)=8, n (DP type 1)=4, n (DP type 2)=5.

FIGS. 9A-9E: IC50 value determination for RG7774 in different cell sub-populations in whole blood of diabetic patients and matched healthy participants. Median Fluorescence intensity (MFI)±SD of CB2 surface expression in diabetic patients (open dots, dotted line) and age-matched healthy participants (closed dots, continuous line) after exposure to increasing concentrations of RG7774. Mean MFI for individual data points is based on duplicate measures FIG. 9A: B-cells (CD45⁺ CD3⁻ CD14⁻ CD19⁺). FIG. 9B: Basophils (CD45⁺ CD3⁻ CD14⁻ CD19⁻ CD193⁺ IgE⁺). FIG. 9C: Monocytes (CD45⁺ CD3⁻ CD14⁺). FIG. 9D: T-cells (CD45⁺ CD3⁺ CD14⁻). FIG. 9E: NK cells (CD45⁺ CD3⁻ CD14⁻ CD19⁻ CD193⁻ IgE⁻ CD16⁺ CD56⁺).

FIG. 10: In vitro IC50 determination of CB2 internalization on B-cells in whole blood from Cynomolgus monkeys. Cynomolgus whole blood was incubated with RG7774 for 1 hour at 37° C. followed by incubation with the anti-human CB2 antibody 1/622. The expression of CB2 on the surface of B-cells was analyzed using a lineage marker mix specific for B-cells and T-cells. The B/T-cell CB2 MFI ratio was calculated and normalized to baseline. 0 μM was set as 100% baseline receptor expression. One Cynomolgus out of five is a non-responder to RG7774 in vitro.

FIG. 11: In vivo feasibility for the new CB2 flow cytometry assay to monitor pharmacodynamic response in vitro on B-cells upon oral dosing. Six healthy Cynomolgus monkeys were dosed with three different doses of an oral micro suspension of RG7774 with two animals per dose group. Blood drawn at different time points was incubated with the anti-human CB2 antibody 1/622. The expression of CB2 on the surface of B-cells was analyzed using a lineage marker mix specific for B-cells and T-cells. The B/T-cell CB2 MFI ratio was calculated and normalized to baseline. 0 μM served as 100% baseline receptor expression. For the animal Mina, blood samples could only be taken up to 1 hour after experimental start, since the animal had to be remove of the study due to a rectal prolapse.

FIG. 12: Single dose in vivo study monitoring the pharmacodynomic effect of RG7774 on CB2 expression on B-cells in Cynomolgus upon oral dosing. Three healthy cynomolgus monkeys were dosed with 100 mg/kg of the CB2 agonist RG7774. The blood draw at different time points was followed by the incubation with the anti-human CB2 antibody 1/622. The expression of CB2 on the surface of B-cells was analyzed using a lineage marker mix specific for B-cells and T-cells. The B/T-cell CB2 MFI ratio was calculated and normalized to baseline. 0 μM served as 100% baseline receptor expression. One out of three cynos is a non-responder to RG7774 in vivo. Due to a storage effect of the antibody working dilution (5 μg/sample) of the anti-human CB2 Ab 1/622 the 4 and 7 hours' time point have been excluded. The working dilution of this antibody is only stable for 2 hours on ice.

FIG. 13: Arithmetic Mean (+SD) Percentage Change-from-Baseline (CFB) of CB2-Receptor Target Engagement on B-cells in Blood versus Time Following Single Oral Dose Administration of 0.75 mg to 300 mg RG7774 in the Fasted State (n=6 on active drug vs 2 on placebo per dose level).

FIG. 14: Arithmetic Mean (+SD) Percentage Change-from-Baseline (CFB) of CB2-Receptor Target Engagement on B-cells in Blood versus Time Following Single Oral Dose Administration of 100 mg RG7774 in fasted and fed state.

FIG. 15: Arithmetic Mean (+SD) Percentage Change-from-Baseline (CFB) of CB2-Receptor Target Engagement on B-cells in Blood versus Time Following Multiple Oral Dose Administration of 20 mg to 300 mg RG7774 once daily for 14 Days.

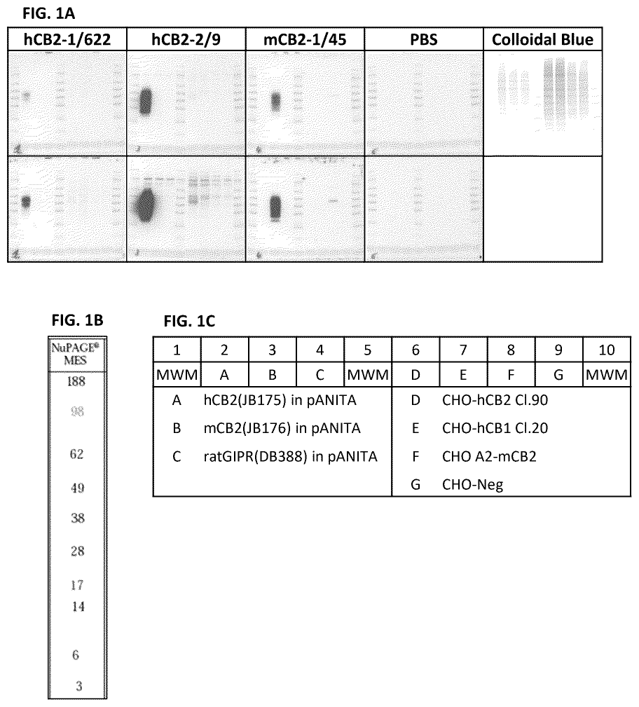

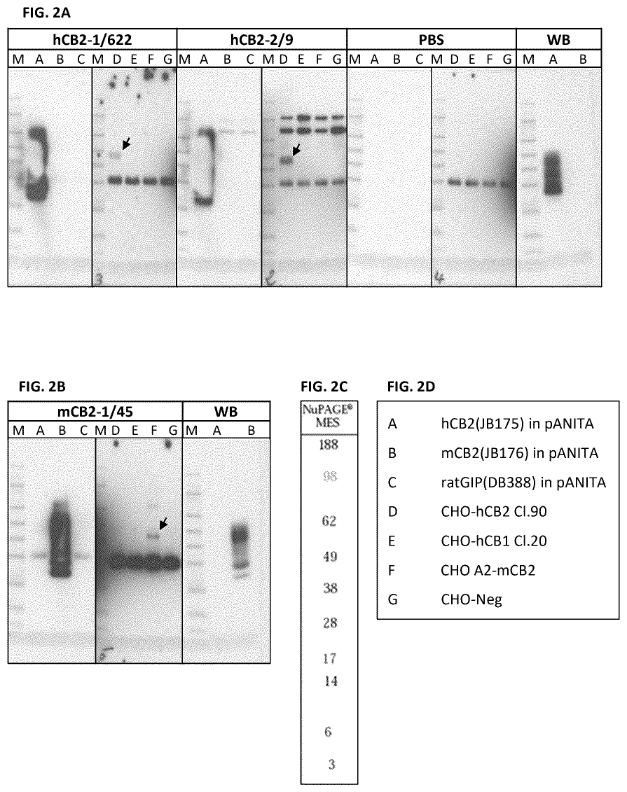

### DETAILED DESCRIPTION OF THE INVENTION

### Definitions

Terms are used herein as generally used in the art, unless otherwise defined in the following.

The term “antibody” herein is used in the broadest sense and encompasses various antibody structures, including but not limited to monoclonal antibodies, polyclonal antibodies, and antibody fragments so long as they exhibit the desired antigen-binding activity.

The term “monoclonal antibody” as used herein refers to an antibody obtained from a population of substantially homogeneous antibodies, i.e. the individual antibodies comprised in the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC, affinity chromatography, size exclusion chromatography) methods. For review of methods for assessment of antibody purity, see, e.g., Flatman et al., J. Chromatogr. B 848:79-87 (2007). In some aspects, the antibodies provided by the present invention are isolated antibodies.

The term “chimeric” antibody refers to an antibody in which a portion of the heavy and/or light chain is derived from a particular source or species, while the remainder of the heavy and/or light chain is derived from a different source or species.

The terms “full-length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N-to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ϵ (IgE), γ (IgG), or μ (IgM), some of which may be further divided into subtypes, e.g. γ1 (IgG₁), γ2 (IgG₂), γ3 (IgG₃), γ4 (IgG₄), α1 (IgA₁) and α2 (IgA₂) for human immunoglobulins. The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding moiety”, “antigen binding domain” or “antigen-binding portion of an antibody” when used herein refers to the part of an antibody that comprises the area which specifically binds to and is complementary to part or all of an antigen. The term thus refers to the amino acid residues of an antibody which are responsible for antigen-binding. An antigen binding domain may be provided by, for example, one or more antibody variable domains (also called antibody variable regions). Particularly, an antigen binding domain comprises an antibody light chain variable region (VL) and an antibody heavy chain variable region (VH). The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N-to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, MD (1991) and/or those residues from a “hypervariable loop”. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementarity determining regions (CDRs). The FR of a variable domain generally consists of four FR domains: FR1, FR2, FR3, and FR4. Accordingly, the HVR and FR sequences generally appear in the following order in VH (or VL): FR1-HCDR1 (LCDR1)-FR2-HCDR2 (LCDR2)-FR3-HCDR3 (LCDR3)-FR4.

Unless otherwise indicated, CDR residues and other residues in the variable domain (e.g., FR residues) are numbered herein according to Kabat et al., supra.

As used herein, the term “antigenic determinant” or “antigen” refers to a site (e.g. a contiguous stretch of amino acids or a conformational configuration made up of different regions of non-contiguous amino acids) on a polypeptide macromolecule to which an antigen binding domain binds, forming an antigen binding domain-antigen complex.

The term “epitope” denotes a protein determinant of an antigen, such as a CB2, capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. The Fc domain of an antibody is not involved directly in binding of an antibody to an antigen. A “Fc domain of an antibody” is a term well known to the skilled artisan and defined on the basis of papain cleavage of antibodies. Depending on the amino acid sequence of the constant region of their heavy chains, antibodies or immunoglobulins of human and mice are divided in the classes: IgA, IgD, IgE, IgG and IgM, and several of these may be further divided into subclasses (isotypes), e.g. IgG₁, IgG₂, IgG₃, and IgG₄, IgA₁, and IgA₂ for human immunoglobulins and IgG₁, IgG₂ₐ, IgG_{2b}, IgG_{2c} and IgG₃ for murine immunoglobulins. According to the heavy chain constant regions the different classes of immunoglobulins are called α(IgA), δ(IgD), ϵ(IgE), γ(IgG), and μ(IgM), respectively.

In one embodiment, the antibodies described herein are of murine IgG class (i.e. of Ig_{G1}, IgG₂ₐ, IgG_{2b}, IgG_{2c} or IgG₃ subclass). In one embodiment, the antibodies described herein are of murine IgG₃ subclass.

By “specific binding” is meant that the binding is selective for the antigen and can be discriminated from unwanted or non-specific interactions. The ability of an antibody to bind to a specific antigen (e.g. CB2) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)).

The term “amino acid” as used within this application denotes the group of naturally occurring carboxy alpha-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program. By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

Antibodies described herein are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NSO cells, SP2/0 cells, HEK293 cells, COS cells, yeast, or E. coli cells, and the antibody is recovered from the cells (from the supernatant or after cells lysis).

Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987).

Expression in NSO cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; Norderhaug, L., et al., J. Immunol. Methods 204 (1997) 77-87. A preferred transient expression system (HEK 293) is described by Schlaeger, E. -J. and Christensen, K., in Cytotechnology 30 (1999) 71-83, and by Schlaeger, E. -J., in J. Immunol. Methods 194 (1996) 191-199.

The heavy and light chain variable domains according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

The term “CB2” or “CB2R” as used herein, refers to any native cannabinoid receptor subtype 2 from any vertebrate source, including mammals such as primates (e.g. humans), non-human primates (e.g. cynomolgus monkeys) and rodents (e.g. mice), unless otherwise indicated. The term encompasses “full-length,” unprocessed CB2 as well as any form of CB2 that results from processing in the cell. The term also encompasses naturally occurring variants of CB2, e.g., splice variants or allelic variants.

The terms “anti-CB2 antibody” and “a-CB2 antibody” refer to an antibody that is capable of specific binding of CB2 with sufficient affinity such that the antibody is useful as a diagnostic and agent in targeting CB2.

By “specific binding” is meant that the binding is selective for the antigen and can be discriminated from unwanted or non-specific interactions. The ability of an antibody to bind to a specific antigen (e.g. CB2) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antibody to an unrelated protein is less than about 10% of the binding of the antibody to the antigen as measured, e.g., by SPR. The antibody comprised in the immunoconjugate described herein specifically binds to CB2.

Conversely, an antibody that “does not bind” to a certain antigen is not capable of binding said antigen with sufficient affinity such that the antibody is useful as a diagnostic agent in targeting said antigen.

The term “cross-reactivity” as used herein refers to the ability of an antibody that binds an antigen of a specific species to also bind the corresponding antigen of another species. For example, an antibody that binds to human CB2 may cross-react with cynomolgus CB2, i.e. bind both human and cynomolgus CB2. Accordingly, an antibody that “does not cross-react” with the corresponding antigen of another species does not bind said antigen. For example, in one aspect the antibody that binds to human CB2 does not cross-react with murine CB2, i.e. binds human CB2 but not murine CB2.

The term “CB2 agonist” refers to compounds that bind to and thereby activate the Cannabinoid Receptor 2. CB2 agonist are described e.g. in WO 2013/068306 Al. “RG7774” refers to the compound(S)-1-[5-tert-Butyl-3-(1-methyl-1H-tetrazol-5-ylmethyl)-3H-[1,2,3] triazolo [4,5-d]pyrimidin-7-yl]-pyrrolidin-3-ol with the formula:

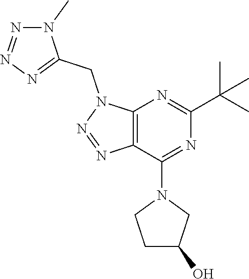 [SMILES: Cn1nnnc1Cn1nnc2c(N3CC[C@H](O)C3)nc(C(C)(C)C)nc21]

### Antibodies Specific for CB2

In one embodiment, the anti-CB2 antibody comprises a heavy chain variable region (VH) comprising a heavy chain complementary determining region (HCDR) 1 of SEQ ID NO: 5, a HCDR 2 of SEQ ID NO: 6, and a HCDR 3 of SEQ ID NO: 7, and a light chain variable region (VL) comprising a light chain complementarity determining region (LCDR) 1 of SEQ ID NO: 8, a LCDR 2 of SEQ ID NO: 9 and a LCDR 3 of SEQ ID NO: 10. In one embodiment, the antibody comprises a heavy chain variable region (VH) comprising an amino acid sequence that is at least about 95%, 96%, 97%, 98%, 99% or 100% identical to the amino acid sequence of SEQ ID NO: 1, and (b) a light chain variable region (VL) comprising an amino acid sequence that is at least about 95%, 96%, 97%, 98%, 99% or 100% identical to the amino acid sequence of SEQ ID NO: 2.

The antibody can be of any type of antibody or fragment thereof that retains specific binding to CB2, particularly human CB2. Antibody fragments include, but are not limited to, Fv molecules, scFv molecule, Fab molecule, and F(ab′)₂ molecules. In particular embodiments, however, the antibody is a full-length antibody. In some embodiments, the antibody comprises an Fc domain, composed of a first and a second subunit. In some embodiments, the antibody is an immunoglobulin, particularly an IgG class, more particularly an IgG₃ subclass immunoglobulin. In one embodiment, the Fc domain is a murine Fc domain.

In some embodiments, the antibody comprises a polypeptide sequence that is at least about 95%, 96%, 97%, 98%, 99% or 100% identical to the amino acid sequence of SEQ ID NO: 3 and a polypeptide sequence that is at least about 95%, 96%, 97%, 98%, 99% or 100% identical to the amino acid sequence of SEQ ID NO: 4. In one embodiment the anti-CB2 antibody is hCB2-1/622.

### CB2 Target Engagement (TE) Assay

Pharmacodynamic biomarkers are often used to evaluate the efficacy of novel molecules and play a crucial role in decision making during the development of novel drugs. Using distal biomarkers it is difficult to assess whether a molecule interacts with its target, as converging signals can influence biomarker response. Whereas target engagement biomarkers proximal to the drug target enable a more robust read-out of the interaction between a drug and its target.

To study pharmacodynamics effects of CB2 agonists the inventors have developed a CB2 agonist target engagement assay. As other members of the G-protein coupled receptor family, activation of CB2 can result in internalization of CB2 from the cell membrane to the endosomal compartment. Thus, changes in cell surface expression of CB2 may present a suitable readout in target engagement. A flow cytometry assay detecting cell surface CB2 expression was investigated. The inventors have demonstrated the suitability of the assay as target engagement biomarker, thereby enabling the study of in vitro and in vivo pharmacodynamic effects of CB2 agonists. The novel assay is a useful tool for pre-clinical and clinical evaluation of CB2 agonist.

Herein disclosed is a method for detecting changes in cell surface CB2 expression in response to the treatment with a CB2 agonist by flow cytometry in a biological samples. The method comprises the a) detecting cell surface CB2 expression levels in a reference sample that has not been treated with a CB2 agonist, b) detecting cell surface CB2 expression in a sample after treatment with a CB2 agonist, and c) comparing the cell surface CB2 expression levels of step b) to those of step a). A decrease in cell surface CB2 expression in response to CB2 agonist treatment is indicative of CB2 target engagement.

Cell surface CB2 expression is detected using an antibody as disclosed herein.

Flow cytometry (FC) is a technique used to detect and measure physical and chemical characteristics, such as the expression of cell surface and intracellular molecules, of a cell population. Detection moieties are employed to stain cell populations expressing the molecules of interest. Flow cytometry methods are described e.g. in Handbook of Flow Cytometry Method, J. Paul Robinson (Editor); Flow Cytometry—A Basic Introduction, Michael G Ormerod (2008) and Current Protocols in Cytometry (2010), Wiley. Commonly antibodies are used for the staining of cell surface proteins. The detection moiety may be conjugated to the antibody that binds to the molecule of interest. Alternatively, the detection moiety may be conjugated to a second antibody. This second antibody in turn binds to the antibody that binds the molecule of interest.

Thus, in the method disclosed herein, the cell surface expression of CB2 may be detected by a) a detection moiety which is conjugated to the anti-human CB2 antibody, or b) a second antibody comprising a detection moiety which binds to the anti-human CB2 antibody. The detection moiety may be a fluorophore.

The assay may be employed to investigate target engagement of diverse CB2agonists. In one embodiment, the CB2 agonist is(S)-1-[5-tert-Butyl-3-(1-methyl-1H-tetrazol-5-ylmethyl)-3H-[1,2,3]triazolo [4,5-d]pyrimidin-7-yl]-pyrrolidin-3-ol.

The biological samples may be obtained from a subject. The subject may be human or cynomolgus monkey. The biological sample may be treated in vitro with the CB2 agonist, i.e. the biological sample is obtained from a subject and exposed to a CB2 agonist. Alternatively, the assay may be performed on biological samples from subjects that have been treated with a CB2 agonist. The biological sample may be whole blood.

The CB2 target engagement assay may also be performed on cell lines expressing CB2.

The assay further enables the determination of dose-response relationships of CB2 agonists. The term “dose-response relationship” or “exposure-response relationship” refers to the relationship between the administered dose or the exposure to a substance in the body, and the measured effect (the response). Target engagement in response to different doses of CB2 agonists may be evaluated. Thus, the assay facilitates the assessment and monitoring of the treatment response to CB2 agonists.

Further, the assay may be used to instruct the dose selection of CB2 agonists. The determination of a dose or exposure-response relationship for a study population may support the dose selection for a target population. The term “target population” as used herein refers to a group of subjects which may be treated with a CB2 agonist. The target population may be defined by characteristics such as disease, disease stage, age, gender etc. The term “study population” as used herein refers to a sample of subjects of the target population which is selected to represent the target population within a study. Hence, findings from the study population shall be applicable to the target population. The disclosed method may be utilized to determine a dose-response relationship of CB2 agonists for a study population thereby informing the dose-selection of the target population.

Moreover, the assay allows to study differences in target engagement between different populations. For example differences in CB2 agonist target engagement in healthy versus diseased, or young versus old subjects may be analyzed. Thus, the assay may support the pre-clinical and/or clinical dose selection for different populations. Also non-responders, i.e. subjects that do not respond to CB2 agonist treatment, may be identified using the disclosed assay.

### EXAMPLES

### Example 1

### Generation of Monoclonal Antibodies Specific for CB2

Our approach utilizes stably transfected mammalian cells (HEK293) expressing recombinant antigens on their cell surface. The transfected cells are also used for measuring seroconversion, hybridoma selection and antibody characterization. By presenting the antigen in its native conformation for immunization and hybridoma selection, this procedure promotes the generation of antibodies capable of binding to the endogenous protein.

The CB2 immunogen was generated by expression of the CB2 N-terminal extracellular domain as a fusion with mouse glycophorin A transmembrane and His-tagged cytoplasmic domains, preceded by a bee-venom melittin signal sequence. This construct enables high expression levels with minimal deleterious effects on the expressing cell; correct orientation of the CB2 fragment relative to the membrane and consequent native posttranslational modifications (e.g. glycosylation). The pANITA3.1 plasmid was used to generate the expression plasmid (based on plasmids described in Dreyer, Beauchamp, Matile & Pluschke BMC Biotechnology 2010, 10:87), the resulting construct transiently transfected into HEK293 cells and cells harvested 2 days post transfection.

Expressed construct sequences are shown in SEQ ID NO: 11 and SEQ ID NO: 12 with CB2-derived fragment underlined. The first 21 residues are removed by cellular signal sequence processing.

| Human_CB2-pANITA3.1 | |
|---|---|
| (SEQ ID NO: 11) | |
| 1 | MKFLVNVALV FMVVYISFIY ASMEECWVTE IANGSKDGLD SNPMKDYMIL |
| 51 | SGPQKAAADY KDDDDKLSPI QHDFPALVMI LIILGVMAGI IGTILLISYC |
| 101 | ISRMTKKSSV DIQSPEGGDN SVPLSSIEQT PNEESSNVSG GHHHHHH |
| Mouse_CB2-pANITA3.1 | |
| (SEQ ID NO: 12) | |
| 1 | MKFLVNVALV FMVVYISFIY ASMEGCRETE VTNGSNGGLE FNPMKEYMIL |
| 51 | SSGQQAAADY KDDDDKLSPI QHDFPALVMI LIILGVMAGI IGTILLISYC |
| 101 | ISRMTKKSSV DIQSPEGGDN SVPLSSIEQT PNEESSNVSG GHHHHHH |

The mice were immunized by repeated intravenous injection of living cells. As soon as the animals showed a specific immune response, the spleen cells were removed for fusion (Köhler, G. & Milstein, C. Continuous cultures of fused cells secreting antibody of predefined specificity. Nature 256, 495-497, 1975).

The positive clones (Table 1) were further characterized by immunofluorescence (IF; Table 2), Western Blot (Table 3, 1) and Immune precipitation (Table 4, 2) on full length hCB2, hCB1, mCB2 stable-transfected CHO-cell lines in addition to the 2 pANITA constructs.

TABLE 1
mAb Overview

| mAb | Antigen | Ig-class | ProtA/G | mg/ml | ml total | Application |
|---|---|---|---|---|---|---|
| hCB2-1/92 | living cells | IgG₂ₐ | 100 ml | 3.5 | 1 | hCB2 + mCB2, CB2_IF |
| hCB2-1/95 | hCB2 (JB175) | IgG₃ | 100 ml | 2.3 | 1 | hCB2 + mCB2, CB2_IF |
| hCB2-1/619 | | IgG₁ | 100 ml | 4.0 | 1 | hCB2, CB2_IF |
| hCB2-1/622 | | IgG₃ | 100 ml | 3.6 | 1 | hCB2, CB2_IF, CB2_WB, |
| | | | | | | CB2_IP, CB2_FC |
| hCB2-1/18 | | IgG₃ | 100 ml | 3.4 | 1 | |
| hCB2-2/9 | | IgG₂b | | | | |
| mCB2-1/12 | living cells | IgG₁ | 100 ml | 2.2 | 1 | |
| mCB2-1/45 | mCB2 | IgG₃ | 100 ml | 1.0 | 1 | |
| mCB2-1/53 | (JB176) | IgG₃ | 100 ml | 1.4 | 1 | |

For IF analysis, methanol-fixed cells were incubated for 20 min in a humid chamber at 37° C. with the respective hCB2 and mCB2 clones. After rinsing twice and washing for 15 min in PBS, 125 μg/ml FITC-conjugated goat anti-mouse IgG antibodies (RAM/IgG (H+L)/FITC, Nordic

Immunological Laboratories) diluted in PBS was added and incubated for 20 min in a humid chamber at 37° C. Cells were rinsed twice and washed for 15 min in PBS, mounted with mounting solution (50% PBS 50% glycerol) and covered with a coverslip. Staining was assessed by fluorescence microscopy on a Leica CTR500 fluorescence microscope and a Leica DFC300 FX digital camera.

TABLE 2
Overview immunofluorescence results

| | | pANITA3.1 | CHODukx | CHODukx | | |
|---|---|---|---|---|---|---|
| | pANITA3.1 | mCB2 | CRE | CRE | CHO-A2 | CHODukx |
| | hCB2 (JB175) | (JB176) | hCB2 Cl.90 | hCB1 Cl.20 | mCB2 | negative |
| hCB2-1/92 | ++ | + | + | − | − | − |
| hCB2-1/95 | ++++ | +++ | − | − | − | − |
| hCB2-1/619 | +++ | − | − | − | − | − |
| hCB2-1/622 | ++++ | − | +++ | − | − | − |
| hCB2-1/18 | +++ | nt | − | − | − | − |
| mCB2-1/12 | nt | ++ | − | − | − | − |
| mCB2-1/45 | nt | ++++ | − | − | +++ | − |
| mCB2-1/53 | nt | ++++ | − | − | − | − |

(“−” no fluorescence detected, “+”-“++++” intensity of fluorescent signal, “nt” not tested)

The antibody hCB2-1/622 demonstrated high fluorescent signal intensities in the two cell lines expressing human CB2 whereas no staining was observed on cells expressing human CB1, mouse CB2 or the negative control (Table 2).

For Western blot analysis, cells were collected using enzyme free dissociation buffer (Cell dissociation buffer enzyme-free Hanks'-based, Gibco), and washed two times with PBS. To prepare cell lysate, 10⁶ cells were lysed with 0.1 ml of lysis buffer (1% NP40, 10% glycerol, 2 mM EDTA, 137 mM NaCl, 20 mM TrisHCl, pH8, Protease Inhibitors) for 10 min on ice. The lysate was cleared by centrifugation at 20′000 g for 5 min. For SDS-PAGE cell lysate was resolved on precast 4-12% gradient gels (NuPAGE® Novex 4-12% Bis-Tris Gel, Invitrogen) with MES running buffer according to the manufacturer's directions. The separation of proteins was visualized by Colloidal Blue staining. The proteins were electrophoretically transferred to nitrocellulose membrane using a dry-blotting system (iBlot, Invitrogen). After blocking the membrane for 16 h in blocking buffer (5% Milk in PBS) at 4° C., specific proteins were detected with appropriate dilutions of mAbs in blocking buffer for 1 h at approximately 20° C. The membrane was then washed four times for 5 minutes in blocking buffer and incubated with horseradish peroxidase conjugated anti-mouse IgG mAb (GAM/IgG (g-chain)/HRP, Sigma, A9917) diluted 1:10'000 in blocking buffer at approximately 20° C. for 1 h. After washing again, blots were developed using ECL Western blotting detection reagents (ECL Western Blotting Substrate, Pierce) to visualize bands.

TABLE 3
Overview Western Blot results

| | pANITA3.1 | pANITA3.1 | pANITA3.1 | CHODukx | CHODukx | | |
|---|---|---|---|---|---|---|---|
| | hCB2 | mCB2 | ratGIPR | CRE | CRE | CHO-A2 | CHODukx |
| | (JB175) | (JB176) | (DB388) | hCB2 Cl.90 | hCB1 Cl.20 | mCB2 | negative |
| hCB2-1/92 | + | − | − | − | − | − | − |
| hCB2-1/95 | ++ | − | − | − | − | − | − |
| hCB2-1/619 | + | − | − | − | − | − | − |
| hCB2-1/622 | +++ | − | − | − | − | − | − |
| hCB2-1/18 | ++++ | ++++ | − | − | − | − | − |
| mCB2-1/12 | +++ | ++ | − | − | − | − | − |
| mCB2-1/45 | ++ | ++++ | − | − | − | + | − |
| mCB2-1/53 | + | ++++ | + | + | − | − | − |
| His-6/9 | +++++ | +++++ | +++++ | − | − | − | − |

(“−” no signal, “+”-“+++++” signal intensity)

Expression of recombinant proteins in pANITA3.1 transfected cells was confirmed by Western blot analysis using anti-His tag antibodies. Cells expressing rat Glucose-dependent insulinotropic polypeptide receptor (GIPR), also a G-protein coupled receptor, were used as a negative control.

The antibody hCB2-1/622 detected a distinct band for hCB2 while no mCB2, hCB1 or rat GIPR was detected (Table 3).

For immunoprecipitation (IP), whole cell lysates were incubated with the respective antibodies. IP was performed according to established protocols and subsequent western blot as described herein above.

TABLE 4
Overview Immunoprecipitation results

| | pANITA3.1 | pANITA3.1 | pANITA3.1 | CHODukx | CHODukx | | |
|---|---|---|---|---|---|---|---|
| | hCB2 | mCB2 | ratGIPR | CRE | CRE | CHO-A2 | CHODukx |
| | (JB175) | (JB176) | (DB388) | hCB2 Cl.90 | hCB1 Cl.20 | mCB2 | negative |
| hCB2-1/92 | + | − | − | − | − | − | − |
| hCB2-1/95 | − | − | − | − | − | − | − |
| hCB2-1/619 | − | − | − | − | − | − | − |
| hCB2-1/622 | ++++ | − | − | + | − | − | − |
| hCB2-1/18 | ++++ | − | − | − | − | − | − |
| mCB2-1/12 | − | + | − | − | − | − | − |
| mCB2-1/45 | − | ++++ | − | − | − | ++ | − |
| mCB2-1/53 | − | ++++ | − | − | − | − | − |

(“−” no signal, “+”-“++++” signal intensity)

The IP experiments demonstrate that the antibody hCB2-1/622 specifically binds hCB2 while no cross reactivity to mCB2 or rat GIPR was found (2 and Table 4).

Further evidence for the specificity of the anti-human CB2 antibody 1/622 was generated by flow cytometry experiments using increasing levels of the antibody on untransfected, hCB1 or hCB2 transfected CHO K1.

The cells were washed 1× with 1× DPBS without CaCl₂ and MgCl₂ (Gibco, #14190-094). Accutase (Sigma, #A6964) was added and the cell were placed back in the incubator. Once the cells had detached, they were collected in a Falcon tube containing DMEM high glucose+GlutaMAX (Gibco, #31966-021) and 10% heat inactivated FBS (Gibco, #16140-071). Cells were centrifuged for 5 minutes at 250×g and 4° C. and the cell pellet was resuspended in cell staining buffer (BioLegend, #420201) and put them on ice until further usage. The cells were incubated with 0.1, 1 or 10 μg/ml hCB2-1/622 antibody and incubated for 30 minutes at 4° C. After the incubation, the tubes were centrifuged for 5 minutes with 250×g and 4° C. The supernatant was removed completely, cells were washed two times with cold cell staining buffer and supernatant removed completely after the centrifugation. Human FcR Blocking Reagent (Miltenyi Biotech, Cat #130-059-901) was diluted according to the package insert instructions with cold cell staining buffer. To each sample 50 μl was added, mixed and incubated for 10 minutes at 4° C. The secondary antibody Goat F(ab′)₂ Anti-Mouse IgG₃ PE (Southern Biotech, Cat #1102-09) was diluted 1:25 in cold cell staining buffer. To each sample 50 μl was added, mixed and incubated for 30 minutes at 4° C. in the dark. After the incubation cells were washed 2× with cold cell staining buffer and supernatant removed completely after centrifugation. 200 μl cold cell staining buffer was added to each tube, mixed carefully and kept on ice or at 4° C. in the dark until acquisition on the Flow Cytometer.

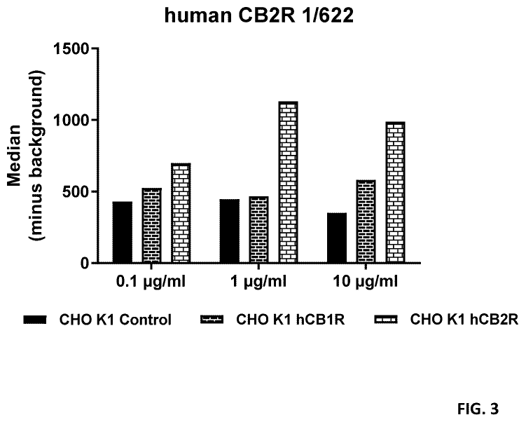

A dose related increase in median fluorescence intensity (MFI) signal was only detected on the CHO K1 h CB2 cell line while the MFI remained at background levels for CHO cells that were either untransfected (CHO K1 controls) or transfected with human CB1 protein (CHO K1 hCB1R; FIG. 3).

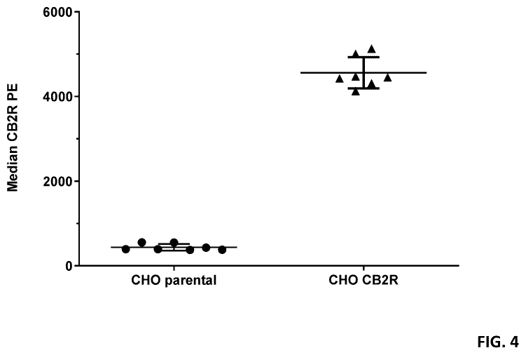

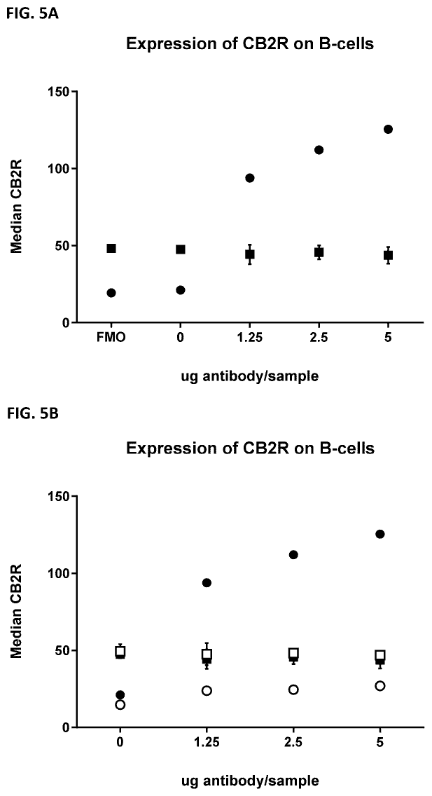

In addition a good signal to noise ratio for the detection of human CB2 in transfected vs parental untransfected CHO-DUXX cells was established by flow cytometry (FIG. 4) further confirming the specificity and suitability of the antibody 1/622 for the specific detection of CB2.

### Example 2

### Comparison of Anti-hCB2 Antibody 1/622 to Commercially Available hCB2 Antibody

Detection of baseline expression of cell surface CB2 on B-cells was assessed for clone 1/622 and compared to a commercially available hCB2 antibody (R&D Systems; clone 352110) (5). The flow cytometry experiment was performed as described in Example 3 yet using untreated whole blood samples and using increasing concentrations of the respective anti-human CB2 antibody.

The dose-dependent increase in Median fluorescence intensity observed only for hCB2-1/622 antibody support a specific binding of this anti-human CB2 antibody in comparison to the comparator and the control conditions without the primary anti CB2 antibody (0 μg/sample) or the FMO (fluorescence minus one) control, in which the secondary fluorescence-labeled antibody is removed.

### Example 3

### Detection of Changes in Surface Expression of CB2 on White Blood Cells by Flow Cytometry Assay

The CB2 is member of the G-protein coupled receptor family which upon ligand (agonist) binding can respond by internalization of the receptor into the endosomal compartment of the cell, thereby removing the respective receptor form the cell surface and initiating downstream signaling effects (Calebiro, D. et al. Best Pract Res Clin Endocrinol Metab 2018:32 (2):83-91). Such changes in cell surface expression of CB2 on B-cells in response to the treatment with the CB2 agonist RG7774 were measured in whole blood samples from nine different healthy human volunteers of working-age. Whole blood samples were treated with eight compound concentrations in duplicates to determine IC50 of RG7774 using a newly developed flow cytometry assay.

Na-Heparin whole blood samples were incubated in a 96-well plate with RG7774 for 1 hour at 37° C. in a humidified incubator with 5% CO₂. Samples were harvested from the 96-well plate and 100 μl were transferred to labeled 5 ml Polystyrene Round Bottom Tubes (Becton Dickinson, Cat #352052). The unlabeled anti-CB2 antibody clone 1/622 was diluted to 50μg/ml with cell staining buffer (BioLegend, Cat #420201). 100 μl diluted anti-CB2 antibody was added to each tube and incubated for 30 minutes at 4° C. For the red blood cell lysis 10× BD PharmLyse lysing buffer (Becton Dickinson, Cat #55899) was diluted to 1× with distilled water and prewarmed to approximately 20° C. After the CB2 antibody incubation 2 ml of prewarmed 1× BD PharmLyse lysing buffer was added to each tube, mixed and incubated for 15 minutes at approximately 20° C. After the incubation, the tubes were centrifuged for 5 minutes at 250×g and 4° C. The supernatant was removed completely, cells were washed two times with cold cell staining buffer and supernatant removed completely after the centrifugation. Human FcR Blocking Reagent (Miltenyi Biotech, Cat #130-059-901) was diluted according to the package insert instructions with cold cell staining buffer. To each sample 50 μl was added, mixed and incubated for 10 minutes at 4° C. The secondary antibody Goat F(ab′)₂ Anti-Mouse IgG₃ PE (Southern Biotech, Cat #1102-09) was diluted 1:25 in cold cell staining buffer. To each sample 50 μl was added, mixed and incubated for 30 minutes at 4° C. in the dark. After the incubation cells were washed 2× with cold cell staining buffer and supernatant removed completely after centrifugation. Lineage marker mix was prepared with cold cell staining buffer containing the antibodies CD3 PerCp-Cy5.5 (BioLegend, Cat #344808), CD14 BV510 clone M5E2 (BioLegend, Cat #301842), CD16 APC (Becton Dickinson, Cat #561304), CD19 BV421 (BioLegend, Cat # 302234), CD45 APC-Cy7 (BioLegend, Cat #304014), CD56 PE-Cy7, clone 5.1.H11 (BioLegend #362510), CD193 FITC (BioLegend, Cat #310720) and IgE Alexa Fluor 647, clone MHE-18(BioLegend #325514). 100 μl lineage marker mix was added to each sample, mixed and incubated for 20 minutes at 4° C. in the dark. After the incubation cells were washed 2× with cold cell staining buffer and supernatant removed completely after the centrifugation. 200 μl cold cell staining buffer was added to each tube, mixed carefully and kept on ice or at 4° C. in the dark until acquisition on the Flow Cytometer.

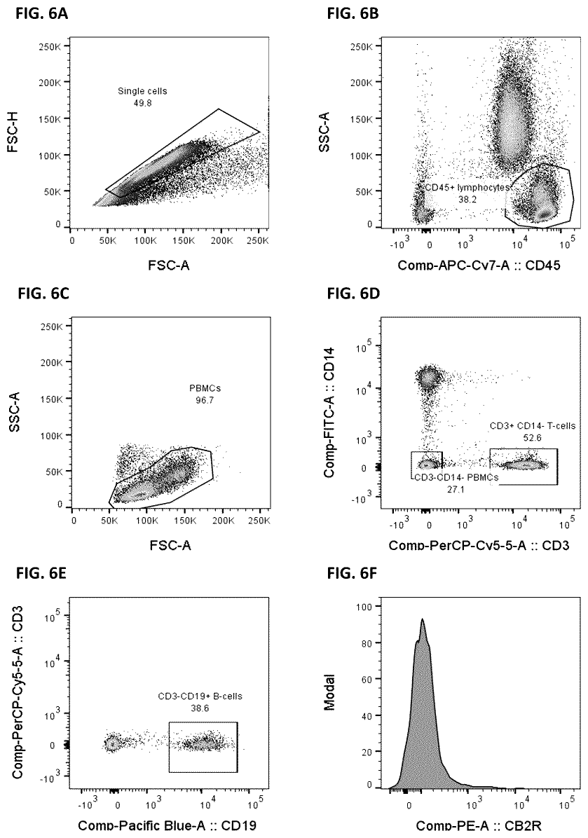

For determination of cell surface expression of CB2 on B-cells, median fluorescent intensity (MFI) was analyzed by gating around the CD45⁺ CD3⁻ CD14⁻ CD19⁺ B-cells (see gating strategy in FIG. 6A to FIG. 6E) and acquiring a minimum of 2000 events in this gate (FIG. 6F). With the lineage marker mix other cell populations like T-cells (CD45⁺ CD3⁺ CD14⁻), Monocytes (CD45⁺ CD3⁻ CD14⁺), Basophils (CD45⁺ CD3⁻ CD14⁻ CD19⁻ CD193⁺ IgE⁺), NK cells (CD45⁺ CD3⁻ CD14⁻ CD19⁻ CD193⁻ IgE⁻ CD16⁺ CD56⁺), Eosinophils (CD45⁺ CD16⁻) and Neutrophils (CD45⁺ CD16⁺) could be analyzed. T-cells were always found to be CB2 negative. Thus, for certain experiments the CD45⁺ CD3⁺ CDC14⁻ T-cells served as an internal negative control population.

To establish IC50 values of RG7774, the ratio of CB2-PE MFI on B-cells to CB2-PE MFI on T-cells normalized to the average of the ratio at 0 μM RG7774 was calculated according to the following equation (1):

$$\frac{B cell to T cell {ratio}_{sample} \times 1}{Avg {( B cell to T cell ratio )}_{o uM}} \qquad ( 1 )$$

Individual IC50 values in healthy human participants for RG7774 range between 64.3 nM and 143.9 nM with an average of 96.5∓SE 73.2-118.7 nM (Table 5).

TABLE 5
IC50 values of RG7774 in working-age healthy participants

| Donor | IC50 [nM] |
|---|---|
| R17.134 | 86.7 |
| R17.135 | 88.2 |
| R18.1 | 115.7 |
| R18.2 | 64.3 |
| R18.3 | 143.9 |
| R18.4 | 98.4 |
| R18.9 | 125.2 |
| R18.13 | 81.6 |
| R18.24 | 64.7 |

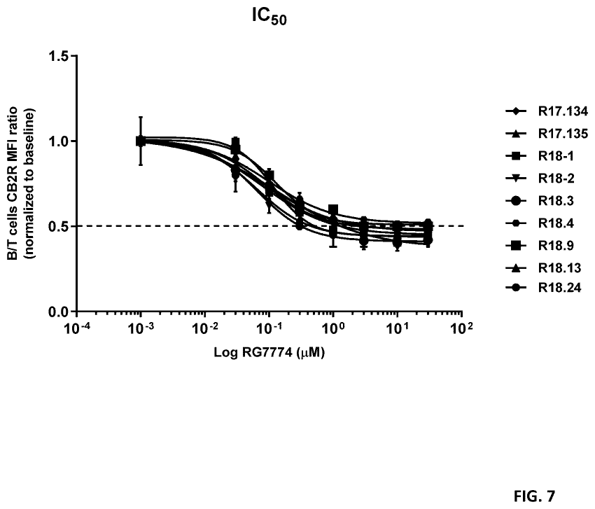

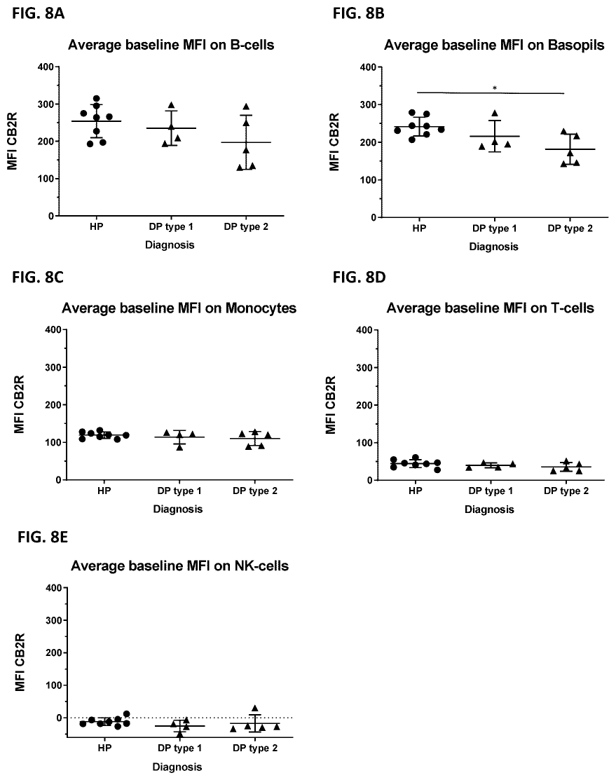

On average, the maximum reduction of CB2 surface expression on B-cells in vitro is 55% over baseline (FIG. 7) and the trend of the curves is comparable and appears similar in both genders (4 female and 5 male donors). The reduction of the CB2 cell surface expression under these experimental conditions can also be detected on Basophils, while it has not been observed on T-cells, Monocytes, NK-cells, Eosinophils and Neutrophils (data not shown).

### Example 4

### CB2 Expression on Different Cell Sub-Populations of Diabetic Patients and Matched Healthy Participants

In order to evaluate whether diabetic disease status of either or of both diabetic types 1 and 2 may have an influence on the expression of CB2 on the surface of circulating blood cells, the CB2 expression levels at baseline (in absence of RG7774) was compared to that on the same cell populations of demographically and age-matched healthy participants (8). The Median fluorescence intensities (MFI) in all participants suggest that CB2 expression is generally low, but that B-cells and Basophils show the highest levels, while monocytes may show around half of the expression and T-cells and NK cells show low or no expression respectively. In addition, this data suggests that diabetic disease status leads to a trend of reduction of CB2 expression on the surface of B-cells and Basophils, although this only reached significance for CB2 expression on Basophils in type 2 diabetics when compared to that in healthy participants (Oneway ANOVA p=0.0245 with Tukey's multiple testing correction p<0.05). For the lower CB2 expression levels on monocytes no such trend was observed.

### Example 5

### IC50 Values of RG7774 in Different Lymphocyte Subsets of Diabetic Patients and Matched Healthy Participants

In order to understand, the exposure-response relationship for a drug-induced CB2 internalization on blood cell populations, in particular on B-cells and Basophils, whole blood of nine different diabetic type 1 and 2 patients and eight demographically and age-matched healthy participants were treated with 8 compound concentrations in duplicates to determine the IC50 of RG7774.

Human whole blood from diabetic patients and age-matched healthy participants was incubated with RG7774 for 1 hour at 37° C. followed by incubation with the anti-human CB2 Ab 1/622. The expression of CB2 on the surface of the different blood cell types was analyzed using the lineage marker mix specific for B-cells, Basophils, T-cells, NK-cells and Monocytes. MFI values at 0 μM RG7774 were set as 100% baseline receptor expression for each cell type.

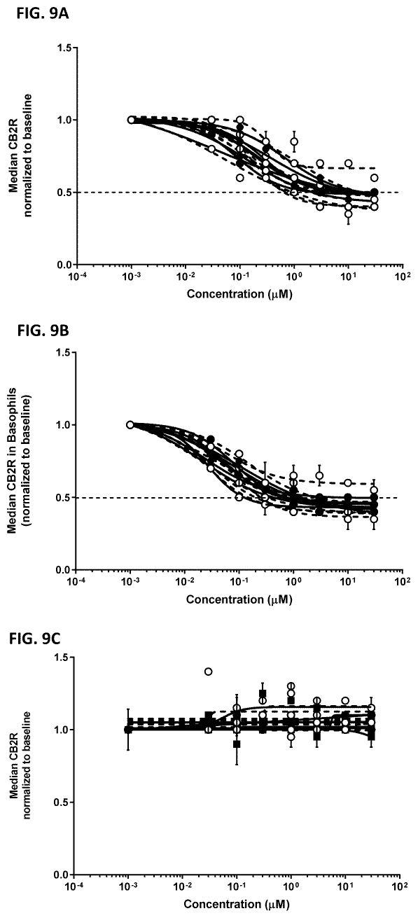

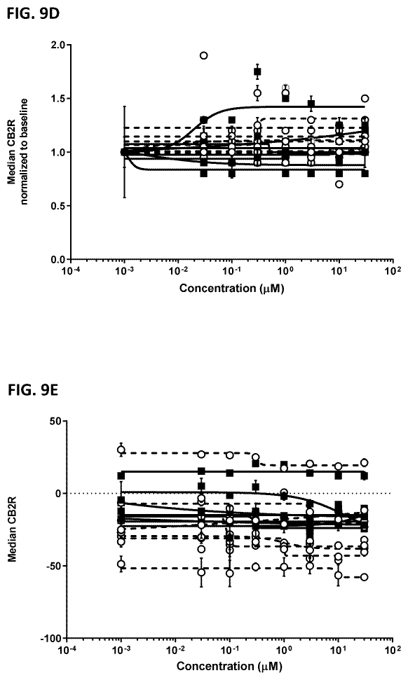

RG7774 induced CB2 internalization was observed on B-cells (FIG. 9A) and Basophils (FIG. 9B), while it was not observed in Monocytes (FIG. 9C), T-cells (FIG. 9D), or NK-cells (FIG. 9E)

This observation was reproducible and the trend of the IC50 curves were comparable in diabetic patients and matched healthy participants, of both genders and both diabetes sub-types (9 and Table 6).

The maximum reduction (Emax) of CB2 surface expression on B-cells and Basophils on average in vitro is ≥50% over baseline in 16 out of 17 patients (Table 6). For B-cells it resulted in an Emax of around ˜56% in diabetics and matched healthy controls, while for Basophils an Emax around ˜60% was observed in both groups on average. Values of subject 774USB16 were excluded since the IC50 on B-cells was an outlier with an about 10× higher value.

TABLE 6
IC50 and Emax values for RG7774 induced CB2 internalization on B-cells and
Basophils from diabetic patients (DM) and matched healthy participants (HP)

| | | Individual & | Individual & Mean | Individual & Mean | Individual & Mean |
|---|---|---|---|---|---|
| | | Mean IC₅₀ (B- | Emax (B-cells) ± | IC₅₀ (Basophils) ± | Emax (Basophils) ± |
| Human | | cells) ± SD per | SD per diagnosis | SD per diagnosis | SD per diagnosis |
| Donor # | Diagnosis | diagnosis (nM) | (%) | (nM) | (%) |
| 7774USB02 | HP | 379.4 | 57.11 | 80.1 | 56.28 |
| 7774USB03 | HP | 689.6 | 52.88 | 114.7 | 66.13 |
| 7774USB05 | HP | 230.2 | 51.18 | 22.1 | 64.04 |
| 7774USB08 | HP | 84.7 | 52.64 | 23.5 | 55.03 |
| 7774USB11 | HP | 43.5 | 70.18 | 26.5 | 57.73 |
| 7774USB12 | HP | 189.3 | 52.40 | 58.1 | 54 |
| 7774USB17 | HP | 135.6 | 60.22 | 55.8 | 58.78 |
| 7774USB19 | HP | 274.0 | 49.16 | 77.3 | 62.7 |
| | Matched HPs | 253.3 ± 206.2 | 55.7 ± 6.8 | 57.2 ± 32.8 | 59.3 ± 4.5 |
| | | (n = 8) | (n = 8) | (n = 8) | (n = 8) |
| 7774USB04 | DM type 1 | 174.6 | 62.05 | 71.96 | 64.9 |
| 7774USB06 | DM type 1 | 225.3 | 56.60 | 38.08 | 68.43 |
| 7774USB14 | DM type 1 | 125.6 | 62.79 | 31.65 | 64.41 |
| (7774USB16) | DM type 1 | (1059.0) | (59.35) | (154.5) | (57.09) |
| | DM type 1 | | 60.5 ± 2.8 | 47.2 ± 21.7 | 65.9 ± 2.2 |
| | | | (n = 3) | (n = 3) | (n = 3) |
| 7774USB01 | DM type 2 | 334.8 | 34.08 | 61.02 | 41.64 |
| 7774USB07 | DM type 2 | 57.9 | 74.13 | 25.72 | 62.91 |
| 7774USB09 | DM type 2 | 121.7 | 50.35 | 54.6 | 54.19 |
| 7774USB10 | DM type 2 | 167.5 | 58.24 | 22.9 | 70.94 |
| 7774USB13 | DM type 2 | 172.3 | 51.25 | 44.36 | 55.48 |
| | DM type 2 | 170.8 ± 102.6 | 53.6 ± 14.5 | 41.7 ± 17.0 | 57.0 ± 10.9 |
| | | (n = 5) | (n = 5) | (n = 5) | (n = 5) |
| | DM (all) | 172.5 ± 82.0 | 56.2 ± 11.7 | 43.8 ± 17.5 | 60.4 ± 9.5 |
| | | (n = 8) | (n = 8) | (n = 8) | (n = 8) |

TABLE 7
Comparison of IC50 (CB2 on B-cells) in healthy working-
age participants (Example 3; Table 5) and diabetic
(DM) or age-matched healthy participants (HP)

| | Mean IC50 (CB2 on | | |
|---|---|---|---|
| | B-cells) ± | Mean Emax ± | Fold change |
| Group/ Diagnosis | SD (nM) | SD (%) | vs HPs |
| Healthy Participants | 96.5 ± 27.1 | 55.0 ± 4.6 | 1 |
| | (n = 9) | (n = 9) | |
| Matched Healthy | 253.3 ± 206.2 | 55.7 ± 6.8 | 2.6 x |
| | (n = 8) | (n = 8) | |
| DM | 172.5 ± 82.0 | 56.2 ± 11.7 | 1.8x |
| | (n = 8) | (n = 8) | |

When comparing the respective mean IC50±SD values of RG774 from diabetic and age-matched healthy controls with the IC50 obtained from working-age healthy participants (data from Example 3; Table 5), the data show a trend that diabetic patients and matched healthy participants may require about 2-3 fold higher exposure of RG774 to achieve half maximum internalization of the CB2 on B-cells relative to working-age healthy participants (Table 7; One-way Anova with Tukeys multiple comparison correction, p=0.0575). The average age of the work-force population is lower than that of the diabetic patients and healthy matched control group.

These in vitro data obtained with the newly developed flow cytometry assay that measures receptor internalization of CB2 on B-cells and other blood cell types in response to RG7774 indicates that the pharmacodynamics response in elderly diabetics and demographically matched healthy participants is shifted to 2-3 times higher concentrations than in young healthy participants, which would suggest that a higher exposure would be required in such participants to achieve a similar drug response effect. This is a key observation that influences dose selection based on age.

### Example 6

### In Vitro Feasibility Assessment of the CB2 Internalization Assay in Cynomolgus Monkeys.

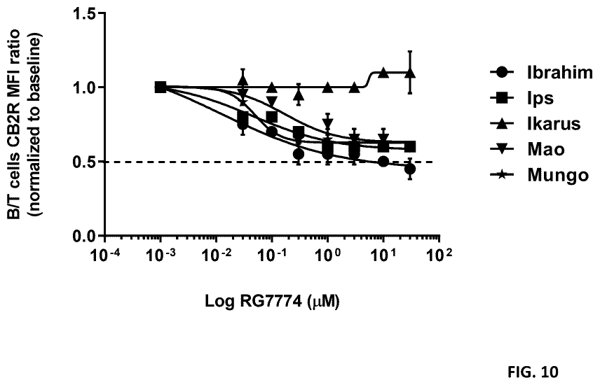

In order to establish whether the CB2 surface assay is cross-reactive in Cynomolgus monkey, blood obtained from five different healthy Cynomolgus monkey has been tested in vitro and the individual IC50 values for RG7774 and maximum reduction of CB2 surface expression in whole blood determined using 8 different compound concentrations in duplicates (FIG. 10, Table 8).

TABLE 8
IC50 (nM) for RG774 in five healthy Cynomolgus monkeys

| Cyno | IC50 (nM) |
|---|---|
| Ibrahim | 12.52 |
| Ips | 44.77 |
| Ikarus | 5445 |
| Mao | 171.2 |
| Mungo | 49.84 |

The maximum reduction of CB2 surface expression on B-cells on average in vitro is 43.8%±7.4% SD over baseline. Noticeably, one of the monkeys (Ikarus) did not show the anticipated pharmacodynamics response within the tested dose range, emphasizing the possibility to identify poor or none-drug responder individuals using this approach. The reduction of the CB2 expression has not been observed in T-cells, Monocytes and Granulocytes (data not shown). This in vitro data confirmed the cross-reactivity of the CB2 flow cytometry assay in Cynomolgus monkeys and justified the experimental testing of the feasibility of this assay to monitor target engagement upon oral dosing in Cynomolgus monkeys in vivo.

### Example 7

### Feasibility Assessment for the Newly Developed CB2 Flow Cytometry Assay to Monitor CB2 Internalization In Vivo Upon Oral Dosing in Cynomolgus Monkeys

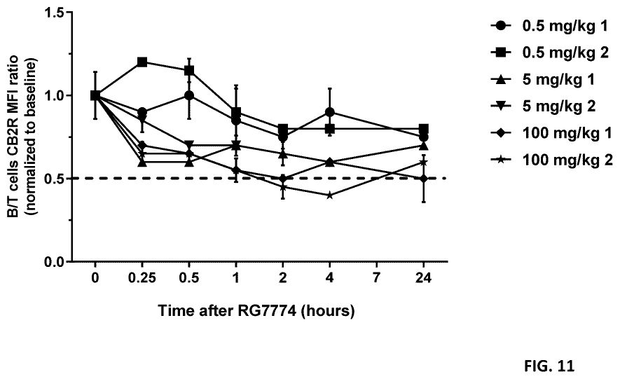

The feasibility of applying the newly developed CB2 flow cytometry assay as an assay to monitor pharmacodynamic response to oral doses of RG7774 was evaluated in three groups of two Cynomolgus monkeys. Upon oral administration of three different single doses of a micro suspension of 0.5, 5 and 100 mg/kg RG7774 respectively, blood samples from all animals were taken at the selected time points: 0 (pre-dose) and 0.25, 0.5, 1, 2, 4, 7 and 24 h post dosing and subjected to the CB2 flow cytometry analysis (FIG. 11). A dose dependent reduction relative to the individual baseline CB2 expression on B-cells was observed that lasted for around 24 hours and enhanced the confidence in the feasibility of the assay for a clinical application.

Based on the increased confidence from the first in vivo study, a second Cynomolgus in vivo study was performed to explore pre-and post-dose time points to better understand the pharmacodynamic responses to RG7774 over time.

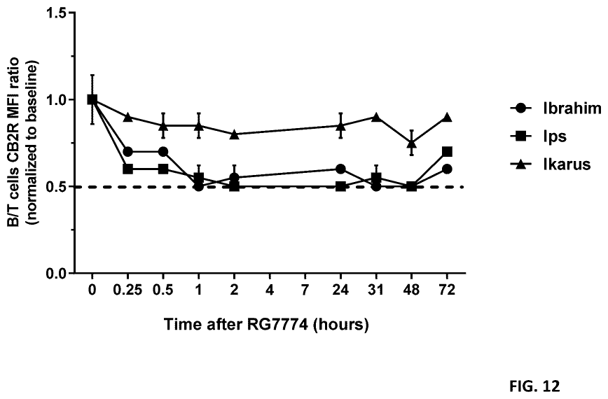

In this in vivo cyno study three cynomolgus monkeys were orally dosed with 100 mg/kg of RG7774. Blood sampling for TE-assay was performed at selected time points: −96, −24 (both time points not shown), 0 (predose) and 0.25, 0.5, 1, 2, 4, 7, 24, 31, 48 and 72 hours post-dosing (FIG. 12).

This study revealed a fast pharamacodynamic effect (within 15 min) and a lasting effect of RG7774 on the surface expression of CB2 on B-cells that could reliably be monitored in a dose dependent fashion over time and has confirmed the utility of the newly developed flow cytometry assay for clinical application as a target engagement assay.

### Example 8

### Successful Implementation of the Assay in Phase I Programs Confirms that the Assay can Use in Clinical Setting—Data Informs Dose Selection for Clinical Development

The clinical utility of the newly developed CB2 flow cytometry assay as a measure for target engagement in a clinical setting was confirmed by its successful implementation in a phase I clinical trial in healthy volunteers. In the clinical setting B/T-cell ratio was not used as it introduced higher variability relative to the baseline normalized B-cell data alone.

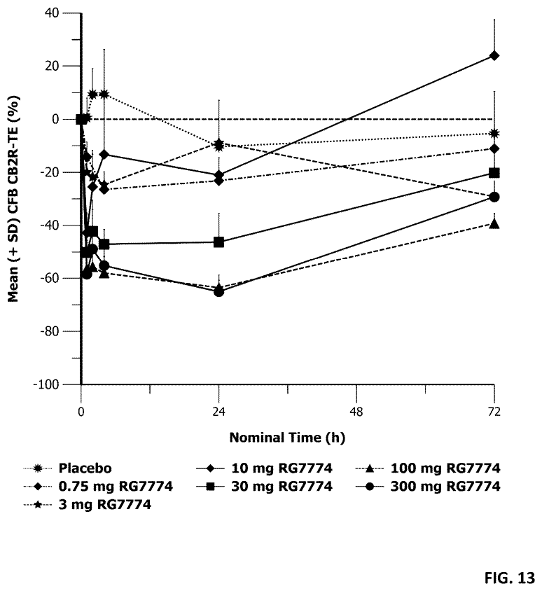

In a first step, oral administration of a single ascending dose (SAD) of RG7774 in the range between 0.75 mg and 300 mg in the fasted state was assessed. Following oral administration of RG774, the onset of CB2 internalization was rapid, with the nadir (lowest CB2 surface expression) achieved at 4 hours post-dose for doses below 10 mg RG7774, and at 1 hour post-dose for doses at 10 mg to 30 mg RG7774 (FIG. 13). For the two highest tested doses of 100 mg and 300 mg RG7774, the time to reach the nadir shifted with the area under the effect curve increasing. The extent of CB2 internalization (i.e. CB2 target engagement) increased dose-dependently, with a marginal increase for doses from 100 mg to 300 mg RG7774. This indicates successful target engagement of RG7774 upon oral dosing. Furthermore, the assay was utilized to inform stopping rules for dose escalation in the SAD part of the first-in-human (FIH) trial.

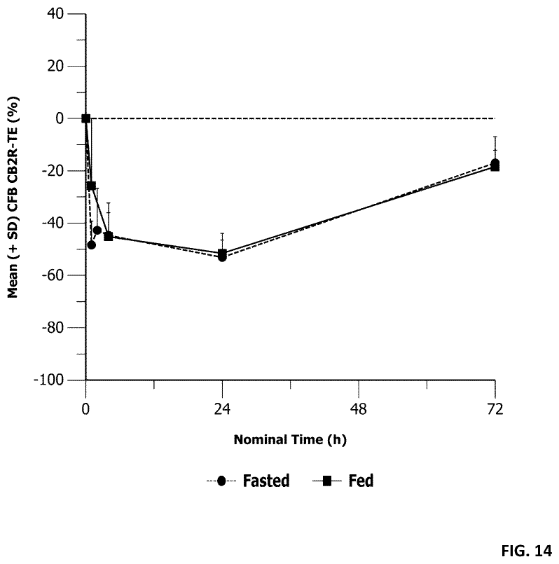

In the second part of the FIH study, the effect of food consumption (FE) was explored by measuring the cell surface CB2 expression by the flow cytometry assay on B-cells in a fasted (n=8) and fed state (n=8) which revealed no relevant impact of food consumption on the pharmacodynamic (PD) effects of RG7774 (FIG. 14).

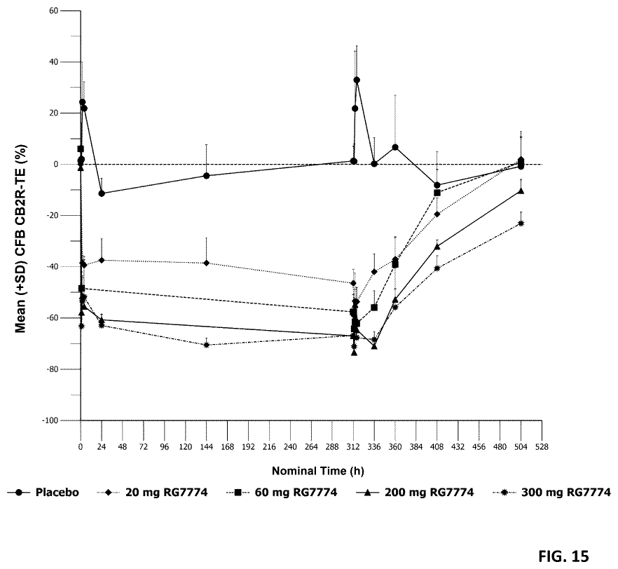

In a third part of the FIH study, the effects of multiple ascending doses (MAD) of 20 mg to 300 mg RG7774 administered once daily over 14 days on CB2 internalization were investigated (FIG. 15).

The newly developed assay helped to characterize the PD effects of CB2 agonists (in this case RG7774) in the clinical setting in healthy volunteers over 14 days of once-daily dosing and showed that the steady-state was reached during the observation period, that over 14 days no desensitization was observed and that the induced drug effect in reduction of CB2 surface receptor expression was reversible roughly within a 1 week upon stopping the drug, although the effect was not yet fully reversed for the two highest doses.

SEQUENCES

| | Amino acid sequence | SEQ ID NO |
|---|---|---|
| Anti-CB2 clone 1/622 | EVQLQQSGPELVKPGASVKISCKTSGYTFTEYTMHWVRQSHGKSLEWIGG | 1 |
| heavy chain variable | IDRYNGDTYYNQKFKDKATLTVDRSSSTAYMELRSLTSEDSAVYFCARNG | |
| region (VH) | AMDYWGQGISVTVSS | |
| Anti-CB2 clone 1/622 light | MKLPVRLLVLMFWIPASSSDVVMTQTPLSLPVSLGDQASISCRSSQSLVH | 2 |
| chain variable region (VL) | SNGNTYLHWFLQKPGQSPKLLIYKVSIRFSGVPDRFSGSGSGTDFTLKIS | |
| | RVEAEDLGVYFCSQSTHVPPLTFGAGTKLELK | |
| Anti-CB2 clone 1/622 full | EVQLQQSGPELVKPGASVKISCKTSGYTFTEYTMHWVRQSHGKSLEWIGG | 3 |
| length heavy chain (HC) | IDRYNGDTYYNQKFKDKATLTVDRSSSTAYMELRSLTSEDSAVYFCARNG | |
| | AMDYWGQGISVTVSSATTTAPSVYPLVPGCSDTSGSSVTLGCLVKGYFPE | |
| | PVTVKWNYGALSSGVRTVSSVLQSGFYSLSSLVTVPSSTWPSQTVICNVA | |
| | HPASKTELIKRIEPRIPKPSTPPGSSCPPGNILGGPSVFIFPPKPKDALM | |
| | ISLTPKVTCVVVDVSEDDPDVHVSWFVDNKEVHTAWTQPREAQYNSTFRV | |
| | VSALPIQHQDWMRGKEFKCKVNNKALPAPIERTISKPKGRAQTPQVYTIP | |
| | PPREQMSKKKVSLTCLVTNFFSEAISVEWERNGELEQDYKNTPPILDSDG | |
| | TYFLYSKLTVDTDSWLQGEIFTCSVVHEALHNHHTQKNLSRSPGK | |
| Anti-CB2 clone 1/622 full | DVVMTQTPLSLPVSLGDQASISCRSSQSLVHSNGNTYLHWFLQKPGQSPK | 4 |
| length light chain (LC) | LLIYKVSIRFSGVPDRFSGSGSGTDFTLKISRVEAEDLGVYFCSQSTHVP | |
| | PLTFGAGTKLELKRADAAPTVSIFPPSSEQLTSGGASVVCFLNNFYPKDI | |
| | NVKWKIDGSERQNGVLNSWTDQDSKDSTYSMSSTLTLTKDEYERHNSYTC | |
| | EATHKTSTSPIVKSFNRNEC | |
| CDR-H1/HVR-H1 of Anti- | EYTMH | 5 |
| CB2 clone 1/622 | | |
| CDR-H2/HVR-H2 of Anti- | GIDRYNGDTYYNQKFKD | 6 |
| CB2 clone 1/622 | | |
| CDR-H3/HVR-H3 of Anti- | NGAMDY | 7 |
| CB2 clone 1/622 | | |
| CDR-L1/HVR-L1 of Anti- | RSSQSLVHSNGNTYLH | 8 |
| CB2 clone 1/622 | | |
| CDR-L2/HVR-L2 of Anti- | KVSIRFS | 9 |
| CB2 clone 1/622 | | |
| CDR-L3/HVR-L3 of Anti- | SQSTHVPPLT | 10 |
| CB2 clone 1/622 | | |
| Human_CB2-pANITA3.1 | MKFLVNVALVEMVVYISFIYASMEECWVTEIANGSKDGLDSNPMKDYMIL | 11 |
| | SGPQKAAADYKDDDDKLSPIQHDFPALVMILIILGVMAGIIGTILLISYC | |
| | ISRMTKKSSVDIQSPEGGDNSVPLSSIEQTPNEESSNVSGGHHHHHH | |
| Mouse_CB2-pANITA3.1 | MKFLVNVALVFMVVYISFIYASMEGCRETEVTNGSNGGLEFNPMKEYMIL | 12 |
| | SSGQQAAADYKDDDDKLSPIQHDFPALVMILIILGVMAGIIGTILLISYC | |
| | ISRMTKKSSVDIQSPEGGDNSVPLSSIEQTPNEESSNVSGGHHHHHH | |

1. An antibody comprising an antigen binding moiety that binds to human cannabinoid receptor subtype 2 (CB2), wherein the antibody comprises a heavy chain variable region (VH) comprising a heavy chain complementary determining region (HCDR) 1 of SEQ ID NO: 5, a HCDR 2 of SEQ ID NO: 6, and a HCDR 3 of SEQ ID NO: 7, and a light chain variable region (VL) comprising a light chain complementarity determining region (LCDR) 1 of SEQ ID NO: 8, a LCDR 2 of SEQ ID NO: 9 and a LCDR 3 of SEQ ID NO: 10.

2. The antibody according to claim 1, wherein the antibody comprises a heavy chain variable region (VH) comprising an amino acid sequence that is at least about 95%, 96%, 97%, 98%, 99% or 100% identical to the amino acid sequence of SEQ ID NO: 1, and (b) a light chain variable region (VL) comprising an amino acid sequence that is at least about 95%, 96%, 97%, 98%, 99% or 100% identical to the amino acid sequence of SEQ ID NO: 2.

3. The antibody according to claim 1, wherein the antibody comprises an Fc domain.

4. The antibody according to claim 3, wherein the Fc domain is an IgG class.

5. The antibody according to claim 3, wherein the Fc domain is a murine Fc domain.

6. The antibody according to claim 1, comprising a polypeptide sequence that is at least about 95%, 96%, 97%, 98%, 99% or 100% identical to the amino acid sequence of SEQ ID NO: 3 and a polypeptide sequence that is at least about 95%, 96%, 97%, 98%, 99% or 100% identical to the amino acid sequence of SEQ ID NO: 4.

7. The antibody according to claim 1, wherein the antibody cross-reacts with cynomolgus CB2.

8. A method for detecting changes in cell surface CB2 expression in response to treatment with a CB2 agonist by flow cytometry in biological samples obtained from a subject, the method comprising:
(a) detecting cell surface CB2 expression levels in a reference sample that has not been treated with a CB2 agonist,
(b) detecting cell surface CB2 expression in a sample after treatment with a CB2 agonist, and
(c) comparing the cell surface CB2 expression levels of step b) to those of step a),
wherein a decrease in cell surface CB2 expression in response to CB2 agonist treatment is indicative of target engagement, wherein cell surface CB2 expression is detected using an antibody comprising an antigen binding moiety that binds to human cannabinoid receptor subtype 2 (CB2), wherein the antibody comprises a heavy chain variable region (VH) comprising a heavy chain complementary determining region (HCDR) 1 of SEQ ID NO: 5, a HCDR 2 of SEQ ID NO: 6, and a HCDR 3 of SEQ ID NO: 7, and a light chain variable region (VL) comprising a light chain complementarity determining region (LCDR) 1 of SEQ ID NO: 8, a LCDR 2 of SEQ ID NO: 9 and a LCDR 3 of SEQ ID NO: 10.

9. A method of assessing treatment response to a CB2 agonist in a subject by flow cytometry, the method comprising:
(a) detecting cell surface CB2 expression levels in a biological sample obtained from said subject before treatment with the CB2 agonist,
(b) detecting cell surface CB2 expression levels in a biological sample obtained from said subject after treatment with the CB2 agonist, and
(c) comparing the cell surface CB2 expression levels of step b) to those of step a),
wherein cell surface CB2 expression is detected using an anti-human CB2 antibody comprising an antigen binding moiety that binds to human cannabinoid receptor subtype 2 (CB2), wherein the antibody comprises a heavy chain variable region (VH) comprising a heavy chain complementary determining region (HCDR) 1 of SEQ ID NO: 5, a HCDR 2 of SEQ ID NO: 6, and a HCDR 3 of SEQ ID NO: 7, and a light chain variable region (VL) comprising a light chain complementarity determining region (LCDR) 1 of SEQ ID NO: 8, a LCDR 2 of SEQ ID NO: 9 and a LCDR 3 of SEQ ID NO: 10.

10. The method according to claim 8 or 9, wherein cell surface expression of CB2 is detected by (a) a detection moiety which is conjugated to the anti-human CB2 antibody, or (b) a second antibody comprising a detection moiety which binds to the anti-human CB2 antibody.

11. The method according to any one of claims 8 and 9, wherein the CB2 agonist is(S)-1-[5-tert-Butyl-3-(1-methyl-1H-tetrazol-5-ylmethyl)-3H-[1,2,3] triazolo [4,5-d]pyrimidin-7-yl]-pyrrolidin-3-ol.

12. The method according to any one of claim 8 and 9, wherein the subject is human or cynomolgus monkey.

13. The method according to any one of claims 8 and 9, wherein the biological samples are treated in vitro with the CB2 agonist.

14. The method according to any one of claims 8 and 9, wherein the subject was treated with the CB2 agonist.

15. The method according to any one of claims 8 and 9, wherein the biological samples are whole blood.

16. The method according to any one of claims 8 and 9, wherein a dose-response relationship of a CB2 agonist is determined.

17. The method according to claim 16, wherein the dose-response relationship of a study population supports the clinical dose selection for a target population.

18. (canceled)

19. (canceled)
